(19) United States
(12) Patent Application Publication
(10) Pub. No.: US 2024/0226233 A1
(43) Pub. Date: Jul. 11, 2024

(54) COMPOSITION COMPRISING ZAG-DERIVED PEPTIDE FOR IMPROVING SCARS AND KELOIDS

(71) Applicant: L&C BIO CO., LTD., Seongnam-si Gyeonggi-do (KR)

(72) Inventors: Dong Hyun Nam, Seoul (KR); Kee Won Lee, Seoul (KR); Hyung Gu Kim, Seoul (KR); Whan Chul Lee, Seoul (KR)

(21) Appl. No.: 18/558,382

(22) PCT Filed: May 3, 2021
(86) PCT No.: PCT/KR2021/005559

§ 371 Date: Nov. 1, 2023

(51) Int. Cl. A61K 38/17; A61K 8/64; A61K 38/10; A61P 17/02; A61Q 19/00

(52) U.S. Cl. CPC A61K 38/1709; A61K 8/64; A61K 38/10; A61P 17/02; A61Q 19/00

(57) ABSTRACT

The present invention relates to a zinc-alpha-2-glycoprotein (ZAG)-derived peptide and a use thereof. The ZAG protein-derived peptide according to the present invention can be used to prevent, treat, or improve scars by suppressing cell migration and reducing the expression of RNA and proteins associated with the formation of scars.

### TECHNICAL FIELD

The present invention relates to a composition including a peptide having an effect of preventing, treating or improving scars.

More particularly, the present invention relates to a pharmaceutical or cosmetic composition for preventing, treating or improving hypertrophic scars or keloids, including a peptide consisting of an amino acid sequence derived from a zinc-alpha-2-glycoprotein (ZAG) protein.

### BACKGROUND ART

A scar is a trace of healed skin that has been damaged by diseases or external irritants. In general, a scar is formed as tissue is repaired when a certain level or more of a wound occurs on/in the skin. When a problem occurs in the process of scarring, the scar may grow excessively. In this case, the scar may not return to the original skin state over time, thereby reducing the quality of life and causing uncomfortable symptoms.

When deep damage occurs in the skin, an excessive amount of collagen accumulates in the affected area, resulting in a larger-than-normal scar. Scars formed in this process are classified as hypertrophic scars or keloids.

Hypertrophic scars are generally caused by excessive accumulation of collagen in the affected area, and the wound heals as the scar becomes larger than the size of the affected area, has a dark color, protrudes, and has a soft shape. These hypertrophic scars occur within a short time after trauma, and may improve over time. Also, the scars occur only within the wound, have a high scarring frequency, and are similar in color to the skin.

On the other hand, a keloid is a disease in which fibrous tissue grows abnormally dense during a wound healing process, and occurs due to problems in the function that regulates wound healing. The most distinctive feature of keloids is a thick dermal layer in which collagen bundles are thick and numerous, so in addition to collagen, an excessive amount of proteoglycans is deposited. Keloid skin poses a cosmetic problem because it spreads not only to the wound area but also the surrounding area, and when keloids occur in oily areas such as the face or joints, they may interfere with the movement of joints or muscles.

A method of treating hypertrophic scars or keloids includes steroids, laser, cryotherapy, surgical excision, microneedle therapy, chemical peels, and the like, treatments using steroids and laser being the most commonly used. Steroids have functions of inhibiting fiber synthesis, reducing inflammation in a scarred area, and inhibiting the production of substances that increase the size of scars. However, to achieve these effects, a high concentration of steroids must be used, which may cause pain during use and produce side effects such as skin atrophy, vasodilation, and the like when used for a long time. Treatment using laser includes a method of making a scar lighter as the epidermis becomes thinner after scabs form on the surface and fall off after the procedure. However, the epidermis becomes thin after the procedure and thus may become sensitive skin, which results in lowered moisture control and immune function and a strong reaction to even minor stimuli.

Meanwhile, the zinc-alpha-2-glycoprotein (ZAG) protein has a function of improving xeroderma or skin barrier dysfunction, and by promoting the expression of filaggrin, it can treat diseases such as xeroderma, skin barrier dysfunction, and inflammatory responses and the like caused by a decrease in filaggrin.

In the present invention, it was confirmed that the ZAG protein may regulate the RNA expression of collagen I, III, and transforming growth factor-beta (TGF-β) and the protein expression of collagen I, III, TGF-β, and pSMAD2/3, which are involved in the formation of scars, preferably hypertrophic scars or keloids, to suppress the formation of scars.

Accordingly, the present invention provides a ZAG protein-derived peptide having an effect of preventing, treating or improving scars, preferably hypertrophic scars or keloids.

Also, the present invention provides a newly discovered core active site (i.e., a fragment) of the ZAG protein-derived peptide having the effect of preventing, treating, or improving scars, preferably hypertrophic scars or keloids.

### RELATED ART DOCUMENT

### [Patent Document]

1. Korean Patent No. 10-2166543

### DISCLOSURE

### Technical Problem

The present invention is directed to providing a zinc-alpha-2-glycoprotein (ZAG) protein-derived peptide capable of preventing, treating or improving scars.

More particularly, the present invention is directed to providing a newly discovered active site of a ZAG protein-derived peptide that suppresses the excessive proliferation and migration of cells in hypertrophic scars or keloids caused by the excessive formation of fibers, and also inhibits the expression of RNA such as collagen I, collagen III, and TGF-β, and the like and the protein expression of collagen I, III, TGF-β, and pSMAD2/3 and providing a composition including the same. The composition may be used as a pharmaceutical or cosmetic composition for preventing, treating or improving scars, preferably hypertrophic scars or keloids.

### Technical Solution

According to one aspect of the present invention, there is provided a pharmaceutical composition for preventing or treating scars, including a ZAG protein-derived peptide.

According to another aspect of the present invention, there is provided a cosmetic composition for improving scars, including a ZAG protein-derived peptide.

### Advantageous Effects

A ZAG protein-derived peptide according to the present invention can have an inhibitory effect on the formation of scars by inhibiting excessive proliferation and migration of cells in scars, preferably hypertrophic scars or keloids caused by excessive formation of fibers.

Also, the ZAG protein-derived peptide can have an effect of preventing, treating or improving scars by inhibiting the expression of RNA and proteins overexpressed in scars, preferably hypertrophic scars or keloids.

In particular, in the present invention, the production costs of a composition, which includes a peptide including a core active site of the selected ZAG protein as an active ingredient, can be reduced by minimizing the length of the peptide.

### DESCRIPTION OF DRAWINGS

FIG. 1 is a graph showing the results of confirming the proliferation of human dermal fibroblasts (HDFs) and keloid fibroblasts (KFs) through a CCK-8 assay when treated with Z1 to Z18 peptides.

FIG. 2 is a graph showing an inhibitory effect on the migration of cells when HDFs cultured in a scar-forming environment created in vitro or KFs cultured in a general medium are scratched and treated with Z16.

FIG. 3 is a graph showing the effect on RNA and protein expression when HDFs cultured in a scar-forming environment created in vitro or KFs cultured in a general medium are scratched and treated with Z16.

FIG. 4 is a graph showing the results of confirming cell proliferation through a CCK-8 assay when oligopeptides are additionally synthesized and HDFs and KFs are then treated with the oligopeptides to identify a core active site of the ZAG-derived peptide.

FIG. 5 is a graph showing an inhibitory effect on the migration of cells when HDFs cultured in a scar-forming environment created in vitro or KFs cultured in a general medium are scratched and treated with Z15mer_1 to Z15mer_4.

FIG. 6 is a graph showing the effect on RNA and protein expression when HDFs cultured in a scar-forming environment created in vitro or KFs cultured in a general medium, are scratched and treated with Z15mer_1 to Z15mer_4.

### BEST MODE

The present inventors have discovered a peptide that is effective in inhibiting the formation of scars, or preventing, treating or improving the scars in the zinc-alpha-2-glycoprotein (ZAG) protein.

Accordingly, the present invention provides a use of the ZAG protein-derived peptide for the preparation of pharmaceuticals or cosmetics for preventing, treating or improving scars.

Also, the present invention provides a pharmaceutical composition for preventing or treating scars, which includes the ZAG protein-derived peptide as an active ingredient, and a cosmetic composition for improving scars, which includes the ZAG protein-derived peptide as an active ingredient.

Further, the present invention provides a method of preventing, treating or improving scars, which includes: administering a therapeutically or physiologically effective amount of the ZAG protein-derived peptide to a subject.

In the present invention, the term “scar” refers to fibrous tissue that replaces normal tissue destroyed by an injury or a disease. Damage to the outer layer of the skin heals by reconstructing the tissue. In this case, scar formation may be minimal. However, when the thick layer of tissue beneath the skin is damaged, reconstruction becomes more complicated. The body has collagen fibers (a protein produced naturally in the body) accumulated therein, which usually results in noticeable scarring.

According to one exemplary embodiment, the scars may include hypertrophic scars, keloids, atrophic scars, or a combination thereof.

In the present invention, the term “hypertrophic scar” refers to a raised scar that occurs when collagen accumulates excessively in an affected area, and remains within the boundaries of a lesion. In general, the hypertrophic scar degenerates naturally after an initial injury. Hypertrophic scars are hard, raised, red in color, itchy, tender, and constricted. Clinically and histologically, hypertrophic scars and keloids are very similar; however, hypertrophic scars enlarge by pushing the scar margins, whereas keloids infiltrate into the surrounding tissue. Hypertrophic scars mature and flatten over time.

In the present invention, the term “keloid” refers to a disease in which fibrous tissue grows abnormally dense during a wound healing process, and occurs due to the problems in the function that regulates wound healing. The fibrous tissue protrudes above the skin surface and extends beyond the boundaries of the original affected area. Keloids are permanent and do not degenerate over time. Keloids are often cosmetically unsightly and may involve pain.

In the present invention, an “atrophic scar” is flat and pressed under the surrounding skin. It is generally small and often round with a serrated or inverted center. Atrophic scar formation may result from surgery, trauma, acne, and chickenpox.

In the present invention, the scars may preferably be hypertrophic scars or keloids caused by excessive formation of fibers.

The term “ZAG protein-derived peptide” used in the present invention refers to an active peptide that exhibits an inhibitory effect on the formation of scars, preferably hypertrophic scars or keloids, in the amino acid sequence of the ZAG protein.

According to one exemplary embodiment, the “ZAG protein-derived peptide” may be a peptide consisting of an amino acid sequence of 10-mer or more and 30-mer or less, which includes a fragment of the ZAG protein. The fragment of the ZAG protein included in the “ZAG protein-derived peptide” may, for example, consist of a continuous amino acid sequence of 10-mer to 30-mer, 10-mer to 25-mer, 10-mer to 20-mer, and 10-mer to 15-mer in the amino acid sequence constituting the ZAG protein.

According to one exemplary embodiment, the fragment of the ZAG protein may consist of a continuous amino acid sequence of 10-mer to 30-mer in the amino acid sequence constituting the ZAG protein.

According to one exemplary embodiment, the fragment of the ZAG protein may consist of any one amino acid sequence selected from SEQ ID NOS: 1 to 18.

According to one exemplary embodiment, the fragment of the ZAG protein may consist of any one amino acid sequence selected from SEQ ID NOS: 19 to 22.

In the present invention, the “ZAG protein-derived peptide” may be used interchangeably with “the fragment of the ZAG protein” in some descriptions. For example, the ZAG protein-derived peptide may consist of any one amino acid sequence selected from SEQ ID NOS: 1 to 18. Alternatively, the ZAG protein-derived peptide may consist of any one amino acid sequence selected from SEQ ID NOS: 19 to 22.

One embodiment of the present invention discloses a ZAG protein-derived peptide that exhibits an inhibitory effect on the formation of scars, preferably hypertrophic scars or keloids. The “ZAG protein-derived peptide” of the present invention may be one of the ZAG protein-derived peptides Z1 to Z18 shown in Table 1 (SEQ ID NOS: 1 to 18, respectively), but the present invention is not limited thereto. These peptides may have an inhibitory effect on scar formation. In particular, it can be seen that the ZAG protein-derived peptides Z1, Z2, Z8 to Z10, Z15, and Z16 have a superior inhibitory effect on the formation of hypertrophic scars or keloids than the ZAG protein, and thus have an excellent effect of preventing, treating or improving hypertrophic scars or keloids.

According to one exemplary embodiment, the “ZAG protein-derived peptide” may be one of the ZAG protein-derived peptides Z15mer_1, Z15mer_2, Z15mer_3, and Z15mer_4 (SEQ ID NOS: 19 to 22, respectively) shown in Table 2.

The Z1 to Z18 peptides or Z15mer_1, Z15mer_2, Z15mer_3, and Z15mer_4 peptides are merely examples of the “ZAG protein-derived peptide” according to the present invention, and the “ZAG protein-derived peptide” may a partial sequence of Z1 to Z18, or Z15mer_1, Z15mer_2, Z15mer_3, and Z15mer_4. Alternatively, the peptide may include an overlapping portion of the respective peptides.

For example, the “ZAG protein-derived peptide” according to the present invention may include an amino acid sequence having 75% or more, preferably 80% or more, more preferably 90% or more, and most preferably 95% or more sequence homology with the amino acid sequence of each of the Z1 to Z18 peptides or the Z15mer_1, Z15mer_2, Z15mer_3, and Z15mer_4 peptides.

Also, the “ZAG protein-derived peptide” may include a cell-permeable peptide, a targeting sequence, a tag, a labeled residue, and an amino acid sequence prepared for the specific purpose of increasing the half-life or stability of the peptide, as well as an amino acid sequence constituting the active peptide, that is, the fragment of the ZAG protein. For example, the present invention may be a fusion peptide in which the cell-permeable peptide, the targeting sequence, the tag, the labeled residue, and the amino acid sequence prepared for the specific purpose of increasing the half-life or stability of the peptide is fused to any one end of one of the Z1 to Z18 peptides or the Z20mer-1, Z20mer-2, Z20mer-3, Z15mer-1, Z15mer-2, Z15mer-3, and Z15mer-4 peptides.

In addition, the “ZAG protein-derived peptide” according to the present invention may also include a functional variant thereof. The functional variant includes biological equivalents of the “ZAG protein-derived peptide” described in the present invention. For example, to further improve the binding affinity and/or other biological characteristics of the peptide, an additional change may be imparted to the amino acid or polynucleotide sequence of the peptide. Such a modification includes deletions, insertions, and/or substitutions of residues in the amino acid sequence of an antibody, and is made based on the relative similarity of amino acid side chain substituents, for example, hydrophobicity, hydrophilicity, or charge size. According to the size, shape, and type of an amino acid side chain substituent, it can be seen that all of arginine, lysine, and histidine are positively-charged residues; alanine, glycine, and serine have similar sizes; and phenylalanine, tryptophan, and tyrosine have similar shapes. Therefore, based on these considerations, arginine, lysine, and histidine; alanine, glycine, and serine; phenylalanine, tryptophan, and tyrosine; and the like may be said to be biologically functional equivalents.

Additionally, the peptide of the present invention may be obtained by various methods widely known in the art. As one example, the peptide may be prepared using polynucleotide recombination and a protein expression system, and may be prepared by an in vitro synthesis method through chemical synthesis such as peptide synthesis, a cell-free protein synthesis method, and the like.

Also, to achieve better chemical stability, enhanced pharmacological properties (half-life, absorbability, potency, efficacy, and the like), modified specificity (e.g., broad biological active spectrum), and reduced antigenicity, a protecting group may be bound to the N- or C-terminus of the peptide. Preferably, the protecting group may include an acetyl group, a fluorenyl methoxy carbonyl group, a formyl group, a palmitoyl group, a myristyl group, a stearyl group or polyethylene glycol (PEG), but may include any component that can modify the peptide, particularly, improve the stability of the peptide, without limitation. The term “stability” used in the present invention means storage stability (e.g., room-temperature storage stability) as well as in vivo stability for protecting the peptide of the present invention from the in vivo attack of a protease.

The “ZAG protein-derived peptide” of the present invention may be included in the pharmaceutical composition or cosmetic composition at 0.0001 to 50% by weight based on the total weight of the composition, but the present invention is not limited thereto. The pharmaceutical composition or cosmetic composition according to the present invention may further contain one or more active ingredients exhibiting the same or similar function, in addition to the ZAG protein-derived peptide.

The ZAG protein-derived peptide of the present invention may be delivered into a subject by a pharmaceutically, physiologically or cosmetologically acceptable carrier such as a colloidal suspension, a powder, saline, a lipid, a liposome, a microsphere, or a nano-spherical particle. These peptides may form a complex with or may be associated with a delivery means, and may be delivered into the body using a delivery system known in the art, such as a lipid, a liposome, a micro-particle, a polymer, a condensation reagent, a polysaccharide, a polyamino acid, a dendrimer, a saponin, an adsorption-enhancing substance or a fatty acid.

In addition, the pharmaceutically, physiologically or cosmetologically acceptable carrier may include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil, and the like, which are commonly used in preparation, but the present invention is not limited thereto. Also, in addition to the above-described components a lubricant, a wetting agent, a sweetening agent, a flavoring agent, an emulsifying agent, a suspending agent, a preservative, and the like may be further included.

It was confirmed that the ZAG protein-derived peptide according to the present invention has an inhibitory effect on the formation of scars by inhibiting the proliferation and migration of cells in scars, preferably hypertrophic scars or keloids, and is also highly effective in preventing, treating, and improving scars by inhibiting the expression of RNA and proteins overexpressed in scars.

Accordingly, the present invention further provides a pharmaceutical or cosmetic composition for preventing, treating or improving scars, including the ZAG protein-derived peptide.

In the present invention, the term “prevention” refers to all actions of inhibiting or delaying scars, preferably “hypertrophic scars” or “keloids” by administration of the pharmaceutical or cosmetic composition including the ZAG protein-derived peptide according to the present invention.

In the present invention, the term “treatment” refers to all actions of alleviating or beneficially changing symptoms of scars, preferably “hypertrophic scars” or “keloids” by administration of the pharmaceutical or cosmetic composition including the ZAG protein-derived peptide according to the present invention.

In the present invention, the term “improvement” refers to all actions of alleviating symptoms of scars, preferably “hypertrophic scars” or “keloids” by administration of the pharmaceutical or cosmetic composition including the ZAG protein-derived peptide according to the present invention.

In the present invention, the term “subject” refers to a subject in need of the prevention, treatment, and improvement of scars, preferably “hypertrophic scars” or “keloids,” and, more specifically, to a mammal such as a human or non-human primate, a mouse, a dog, a cat, a horse, a cow, and the like.

The ZAG protein-derived peptide may be parenterally or orally administered to a subject. Because the ZAG protein-derived peptide may be used for preventing, treating or improving scars, preferably “hypertrophic scars” or “keloids,” the ZAG protein-derived peptide may be prepared into a pharmaceutical or cosmetic composition that may be applied parenterally, particularly, to the skin, and topically administered, but the present invention is not limited thereto. For example, the ZAG protein-derived peptide may be formulated into dosage forms such as ointments, gels, creams, lotions, and the like. The type of topical administration may be, for example, application onto the skin, transdermal penetration using microneedles, intradermal injection, and the like, but the present invention is not limited thereto. For example, the composition may be preferably applied on the skin or a patch formulation including the composition may be preferably attached onto the skin.

The composition may further include a pharmacologically or physiologically acceptable carrier, excipient or diluent, as well as the ZAG protein-derived peptide described above for administration. Examples of the suitable carrier, excipient, and diluent that may be included in the composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, mineral oil, and the like. When the composition is formulated, the composition may further include conventional fillers, extenders, binders, disintegrants, surfactants, anti-coagulants, lubricants, wetting agents, fragrances, emulsifying agents, preservatives, and the like.

The formulation of the composition may include a solid, an emulsion (including a microemulsion), a suspension, a cream, a lotion, a gel, a powder, or other typical solid or liquid compositions used to be applied to the skin and other tissues to which the composition of the present invention may be applied. Such a composition may additionally include an antimicrobial agent, a moisturizing agent, a hydration agent, a penetration agent, a preservative, an emulsifying agent, a natural or synthetic oil, a solvent, a surfactant, a detergent, a gelling agent, an emollient, an antioxidant, a fragrance, a filler, a thickener, a wax, a deodorant, a dyestuff, a colorant, a powder, a viscosity-controlling agent, and water, and may optionally include an anesthetic, an anti-itching active agent, a botanical extract, a conditioning agent, a darkening or lightening agent, glitter, a humectant, mica, a mineral, a polyphenol, silicone or a derivative thereof, a sunblock, a vitamin, and phytomedicine.

One embodiment of the present invention may provide a cosmetic composition including the ZAG protein-derived peptide. More specifically, the cosmetic composition may be a cosmetic composition for improving scars, preferably hypertrophic scars or keloids. The cosmetic composition may have a formulation selected from the group consisting of a suspension, an emulsion, a gel, and a paste, but the present invention is not limited thereto.

The dose of the composition varies according to the body weight, age, sex, health condition, and diet of a subject, an administration time, an administration method, an excretion rate, the severity of a disease, and the like. A daily dose may be approximately 0.001 to 100 mg/kg, for example, 0.01 to 10 mg/kg, but the present invention is not limited thereto. The composition may be administered once daily or in divided doses

The present invention provides a method of preventing, treating or improving scars, preferably hypertrophic scars or keloids, which includes: administering a therapeutically or physiologically effective amount of the ZAG protein-derived peptide to a subject.

The present invention will be described in more detail with reference to the following embodiments thereof. However, it should be understood by those skilled in the art that the following embodiments are not intended to limit the scope of the present invention, and various modifications, variations or applications are possible without departing from the technical details derived from the details described in the appended claims.

### MODE FOR INVENTION

### Examples

### Preparation Example 1: Synthesis, Separation and Purification of Peptides

To discover peptides including an active site in a ZAG protein, 18 peptide candidates (Z1 to Z18, each having SEQ ID NOS: 1 to 18) were chosen so that 15 amino acids in the entire 298 amino acid sequence (GenBank: AAH05306.1) (SEQ ID NO: 23) overlapped. Each of the peptides was manufactured through a solid-phase peptide synthesis method.

It was confirmed through HPLC analysis that the peptides synthesized in the present invention had a purity of 90% or more. The molecular weights of the purified peptides were determined through mass spectrometry.

Table 1 below lists peptide candidates and amino acid sequences used to discover the active site of the ZAG protein.

TABLE 1

| | Molecular | | |
|---|---|---|---|
| Peptide | weight | | SEQ ID |
| name | (g/mol) | Sequence | NO: |
| Z1 | 3443.74 | QENQDGRYSLTYIYTGLSKHVEDVPAFQAL | 1 |
| Z2 | 3589.04 | GSLNDLQFFRYNSKDRKSQPMGLWRQVEGM | 2 |
| Z3 | 3751.07 | EDWKQDSQLQKAREDIFMETLKDIVEYYND | 3 |
| Z4 | 3485.73 | SNGSHVLQGRFGCEIENNRSSGAFWKYYYD | 4 |
| Z5 | 3520.96 | GKDYIEFNKEIPAWVPFDPAAQITKQKWEA | 5 |
| Z6 | 3604.16 | EPVYVQRAKAYLEEECPATLRKYLKYSKNI | 6 |
| Z7 | 3370.85 | LDRQDPPSVVVTSHQAPGEKKKLKCLAYDF | 7 |
| Z8 | 3344.62 | YPGKIDVHWTRAGEVQEPELRGDVLHNGNG | 8 |
| Z9 | 3296.64 | TYQSWVVVAVPPQDTAPYSCHVQHSSLAQP | 9 |
| Z10 | 3396.73 | GLSKHVEDVPAFQALGSLNDLQFFRYNSKD | 10 |
| Z11 | 3661.07 | RKSQPMGLWRQVEGMEDWKQDSQLQKARED | 11 |
| Z12 | 3535.93 | PFDPAAQITKQKWEAEPVYVQRAKAYLEEE | 12 |
| Z13 | 3457.97 | CPATLRKYLKYSKNILDRQDPPSVVVTSHQ | 13 |
| Z14 | 3478.88 | IFMETLKDIVEYYNDSNGSHVLQGRFGCEI | 14 |
| Z15 | 3691.00 | ENNRSSGAFWKYYYDGKDYIEFNKEIPAWV | 15 |
| Z16 | 3420.95 | APGEKKKLKCLAYDFYPGKIDVHWTRAGEV | 16 |
| Z17 | 3306.57 | QEPELRGDVLHNGNGTYQSWVVVAVPPQDT | 17 |
| Z18 | 3215.56 | AVPPQDTAPYSCHVQHSSLAQPLVVPWEAS | 18 |

### Example 1: Peptide Selection Using CCK-8 Assay

A CCK-8 assay was performed to select amino acid sequences corresponding to the core active region of ZAG, which has an inhibitory effect on the formation of scars, among the 18 peptide candidates. Human dermal fibroblasts (HDFs) and keloid fibroblasts (KFs) were treated with the ZAG protein-derived peptide candidates (Z1 to Z18) and the ZAG protein (ZAG) at a concentration of 10 μg/mL, and cell proliferation was then confirmed using a CCK-8 assay. A group treated with the ZAG protein alone was used as a positive control, and an untreated group was used as a negative control.

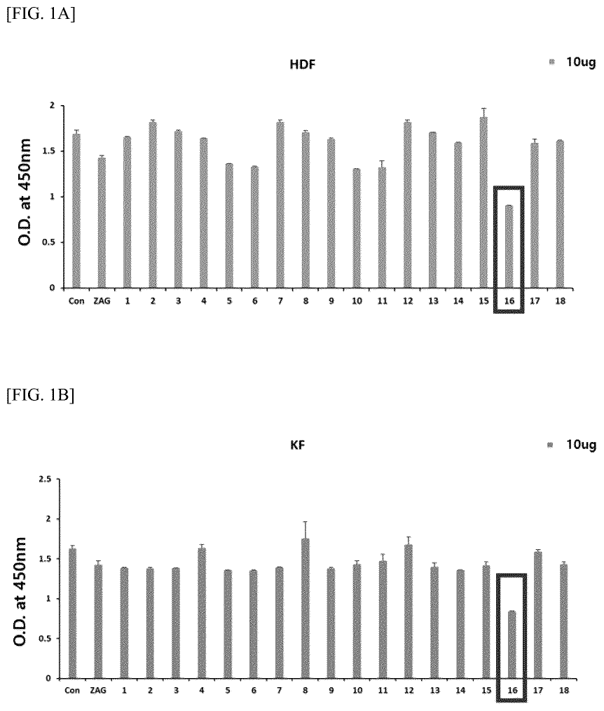

The results of confirming cell proliferation are shown in FIG. 1.

FIG. 1 is a graph showing the results of confirming the proliferation of human dermal fibroblasts (HDFs) and keloid fibroblasts (KFs) through a CCK-8 assay when treated with Z1 to Z18 peptides.

As shown in FIG. 1, the positive control (ZAG) treated with the ZAG protein alone showed decreased cell proliferation in both HDF and KF compared to the untreated negative control (Con).

In particular, it can be seen that, among the peptide candidates, the Z16 peptide (16) showed the lowest cell proliferation in both HDF and KF. From these results, it can be confirmed that Z16 is a substance corresponding to the core active area that has an inhibitory effect on the formation of hypertrophic scars or keloids.

### Example 2-1: Confirmation of Inhibition of Hypertrophic Scar or Keloid Formation Using Scratch Assay

HDFs and KFs were seeded in a 6-well plate. After 24 hours, the center of each well was scratched to create a wound. Thereafter, the HDF test group was treated with 10 ng/mL of TGF-β, which is overexpressed during scar formation, to create a scar environment, and then treated with the selected Z16 peptide. The migration of cells in the affected area was confirmed 24 hours later.

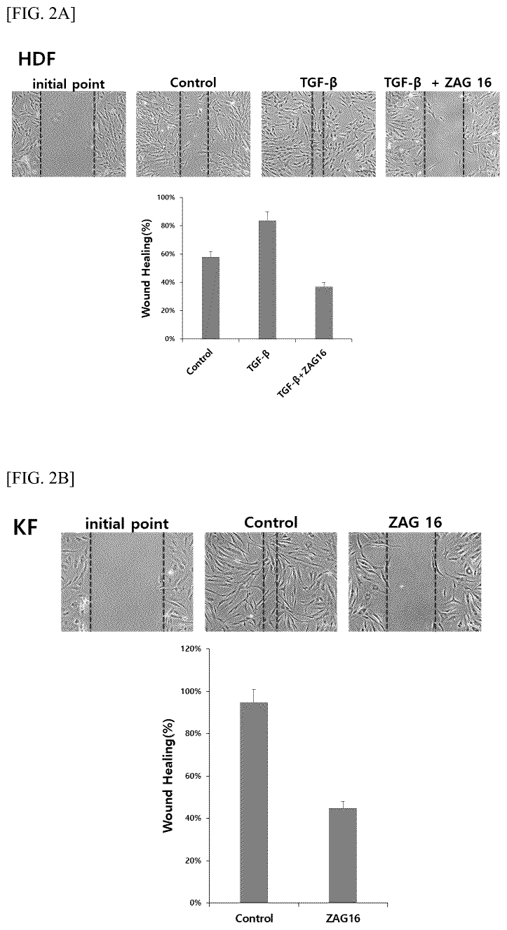

FIG. 2 is a graph showing an inhibitory effect on the migration of cells when HDFs cultured in a scar-forming environment created in vitro or KFs cultured in a general medium are scratched and treated with Z16.

In the case of HDF, it can be seen that most of the affected area was filled with cells due to cell migration in the TGF-β-treated group (TGF-β) compared to the untreated group (control), but the migration of these cells was inhibited in the group (TGF-β+ZAG16) treated with TGF-β and Z16 (FIG. 2A).

Also, in the case of KF, it can be seen that the migration of cells in the affected area was inhibited in the Z16-treated group (ZAG 16) (FIG. 2B). Based on these results, it can be seen that Z16 is involved in not only cell proliferation but also cell migration, and thus is effective in suppressing the formation of hypertrophic scars and keloids.

### Example 2-2: Confirmation of Inhibition of Protein and RNA Expression Associated with Formation of Hypertrophic Scars or Keloids

Cells were treated with Z16 under the same conditions as in Example 2-1, and the expression of RNA and proteins involved in the formation of hypertrophic scars or keloids was analyzed. The expression was confirmed through PCR and Western blotting.

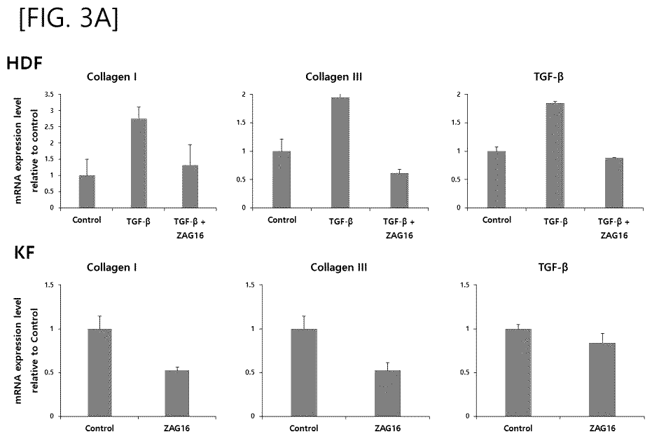

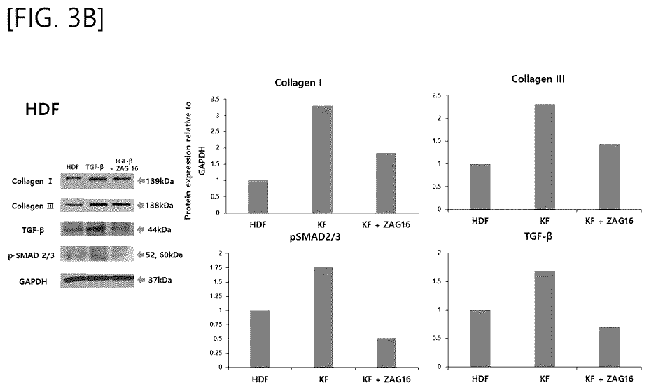

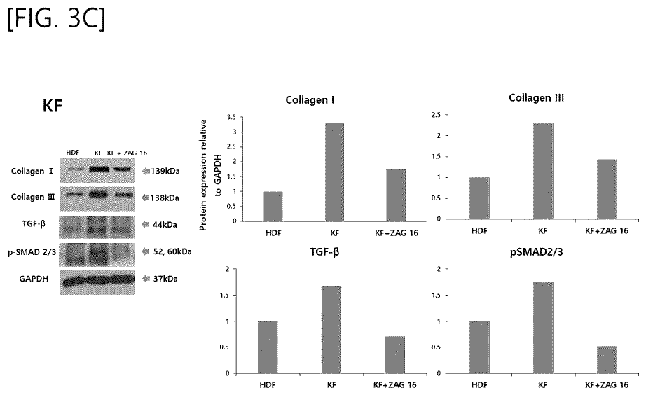

FIG. 3 is a graph showing the effect on RNA and protein expression when HDFs cultured in a scar-forming environment created in vitro or KFs cultured in a general medium are scratched and treated with Z16. Here, FIG. 3A and FIGS. 3B and 3C represent the expression of RNA and the expression of proteins, respectively.

In the case of HDF, it can be seen that the mRNA expression of collagen I, III, and TGF-β and the protein expression of collagen I, III, TGF-β, and pSMAD2/3 increased in the TGF-β-treated group (TGF-β) compared to the untreated negative control (the control in FIG. 3A and HDF in FIG. 3B), but the mRNA and protein expression decreased in the group (TGF-β+ZAG-16) treated with TGF-β and Z16.

Also, in the case of KF, it can be seen that the mRNA expression of collagen I, III, and TGF-β and the protein expression of collagen I, III, TGF-β, and pSMAD2/3, which were expressed at higher levels than HDF in the untreated negative control (the control in FIG. 3A and KF in FIG. 3C), decreased in the Z16-treated group (KF+ZAG). Based on these results, it can be seen that Z16 suppressed the expression of RNA and proteins associated with the formation of hypertrophic scars or keloids.

### Preparation Example 2: Additional Synthesis of ZAG-Derived Peptides

Based on the ZAG16 peptide (Z16) selected as the core active site of ZAG having an inhibitory effect on the formation of scars (hypertrophic scars or keloids) from the eighteen peptide candidates (Z1 to Z18), Z16-derived oligopeptides were additionally synthesized to identify the core active site (Table 2). Each of the peptides in Table 2 was manufactured by a solid-phase peptide synthesis method. It was confirmed through HPLC analysis that the peptides synthesized in the present invention had a purity of 90% or more. The molecular weights of the purified peptides were determined through mass spectrometry.

Table 2 below lists peptide candidates and amino acid sequences used to discover the active site of the ZAG protein.

TABLE 2

| | Molecular | | |
|---|---|---|---|
| Peptide | weight | | SEQ ID |
| name | (g/mol) | Sequence | NO: |
| Z15mer_1 | 1711.03 | APGEKKKLKCLAYDF | 19 |
| Z15mer_2 | 1787.17 | KKLKCLAYDFYPGKI | 20 |
| Z15mer_3 | 1825.02 | LAYDFYPGKIDVHWT | 21 |
| Z15mer_4 | 1728.16 | YPGKIDVHWTRAGEV | 22 |

### Example 3: Confirmation of Active Site of ZAG-Derived Peptide Using CCK-8 Assay

A CCK-8 assay was performed to select an amino acid sequence corresponding to the core active region of Z16, which has an inhibitory effect on the formation of hypertrophic scars or keloids.

Specifically, HDFs and KFs were treated with the ZAG protein (ZAG), Z16, and the ZAG protein-derived peptide candidates (Z15mer_1 to Z15mer_4) additionally synthesized in Preparation Example 2 at a concentration of 10 μg/mL, and cell proliferation was then confirmed through a CCK-8 assay.

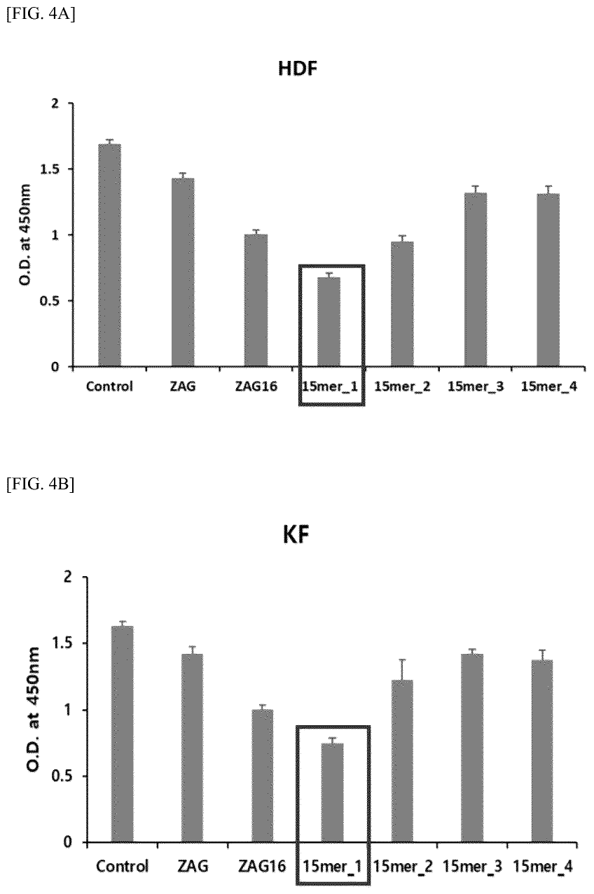

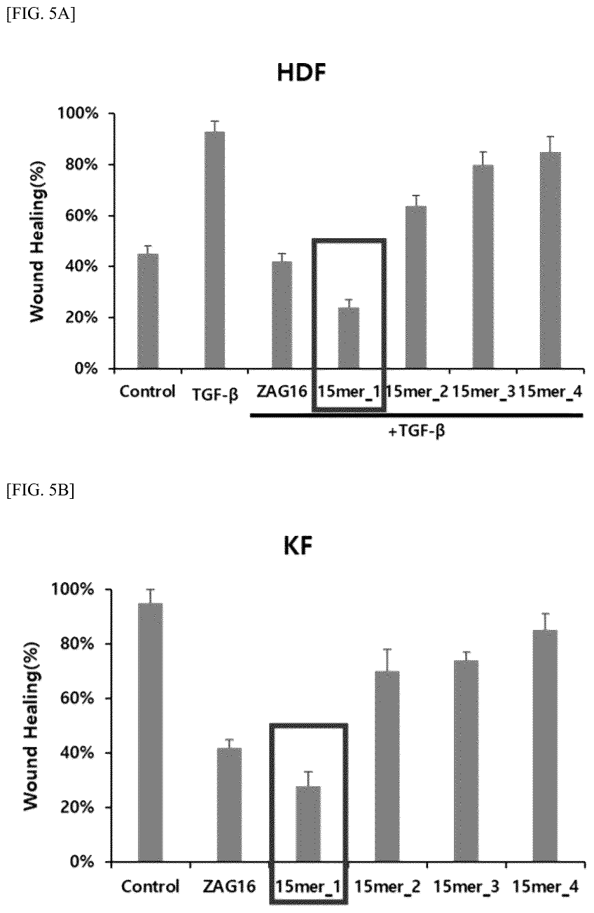

FIG. 4 is a graph showing the results of confirming cell proliferation through a CCK-8 assay when oligopeptides are additionally synthesized and HDFs and KFs are then treated with the oligopeptides to identify a core active site of the ZAG-derived peptide.

As shown in FIG. 4, it can be seen that, among the oligopeptide candidates, the test group treated with Z15mer-1 showed the lowest proliferation in both the HDF and KF test groups. Through this, it can be confirmed that Z15mer-1 is a substance corresponding to the core active region of Z16. Based on these results, it can be seen that Z15mer-1 is a substance corresponding to the core active region of Z16.

### Example 4-1: Confirmation of Inhibition of Hypertrophic Scar or Keloid Formation Using Scratch Assay

HDFs and KFs were seeded in a 6-well plate. After 24 hours, the center of each well was scratched to create a wound generation model. Thereafter, the HDF test group was treated with 10 ng/mL of TGF-β, which is overexpressed during scar formation, to create a scarring environment, and then treated with Z15mer_1 to Z15mer_4 additionally synthesized in Preparation Example 2. The migration of cells in the affected area was confirmed 24 hours later.

FIG. 5 is a graph showing an inhibitory effect on the migration of cells when HDFs cultured in a scar-forming environment created in vitro or KFs cultured in a general medium are scratched and treated with Z15mer_1 to Z15mer_4.

In the case of HDF, it can be seen that the migration of cells in the affected area was inhibited when TGF-β was treated with Z16, Z15mer_1 to Z15mer-4, compared to the TGF-β-treated group (TGF-β). In particular, it can be seen that the test group (15mer_1) treated with TGF-β and Z15mer_1 showed the greatest inhibition of cell migration in the affected area.

Also, in the case of KF, it can be seen that the test group (15mer_1) treated with Z15mer_1 showed the greatest inhibition of cell migration in the affected area. Based on these results, it can be seen that Z15mer_1 corresponding to the core active region of Z16 inhibited the formation of hypertrophic scars and keloids more effectively than ZAG16.

### Example 4-2: Confirmation of Inhibition of Protein and RNA Expression Associated with Formation of Hypertrophic Scars or Keloids

Cells were treated with Z16 under the same conditions as in Example 3-1, and the expression of RNA and proteins involved in the formation of hypertrophic scars or keloids was analyzed. The expression was confirmed through PCR and Western blotting.

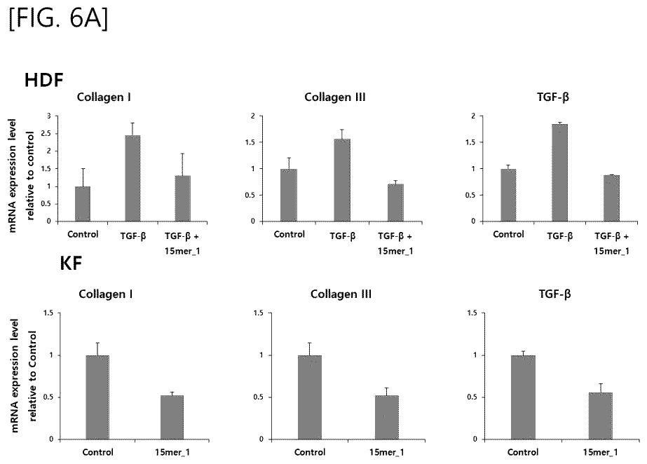

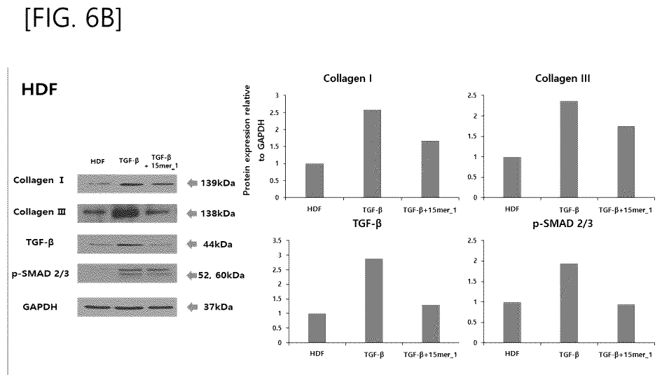

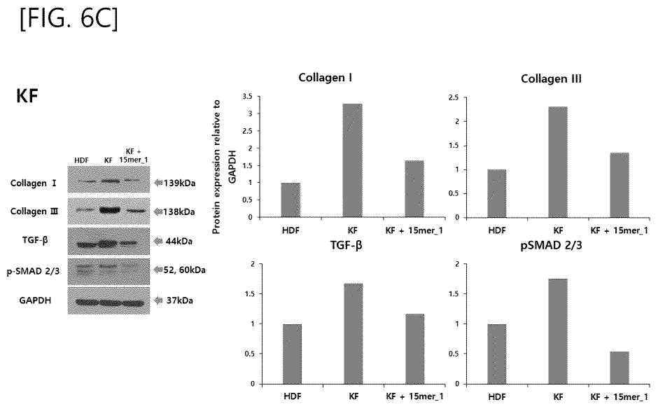

FIG. 6 is a graph showing the effect on RNA and protein expression when HDFs cultured in a scar-forming environment created in vitro or KFs cultured in a general medium are scratched and treated with Z15mer_1 to Z15mer_4.

In the case of HDF, it can be seen that the mRNA expression of collagen I, III, and TGF-β and the protein expression of collagen I, III, TGF-β, and pSMAD2/3 decreased in the group (TGF-β+15mer_1) treated with TGF-β and Z15mer_1 compared to the TGF-β-treated group (TGF-β).

Also, in the case of KF, it can be seen that the collagen I, III, and TGF-β mRNA and the collagen I, III, TGF-β, and pSMAD2/3 proteins were expressed in the untreated negative control (the control in FIG. 6A and KF in FIG. 6C) at higher levels than HDF, but the mRNA and protein expression decreased in the Z15mer_1-treated group (KF+15mer_1). Based on these results, it can be seen that Z15mer_1 inhibited the expression of RNA and proteins associated with the formation of hypertrophic scars or keloids to the same extent as ZAG16 (Z16).

### INDUSTRIAL APPLICABILITY

The ZAG protein-derived peptide according to the present invention may have an effect of inhibiting the formation of scars by inhibiting excessive proliferation and migration of cells in scars, preferably hypertrophic scars or keloids caused by excessive formation of fibers.

Also, the ZAG protein-derived peptide may have an effect of preventing, treating or improving scars by inhibiting the expression of RNA and proteins overexpressed in scars, preferably hypertrophic scars or keloids.

1. A pharmaceutical composition for preventing or treating scars, comprising a zinc-alpha-2-glycoprotein (ZAG) protein-derived peptide.

2. The pharmaceutical composition of claim 1, wherein the scars include hypertrophic scars, keloids, atrophic scars, or a combination thereof.

3. The pharmaceutical composition of claim 1, wherein the ZAG protein-derived peptide is a peptide consisting of an amino acid sequence of 10-mer or more and 30-mer or less, which includes a fragment of the ZAG protein.

4. The pharmaceutical composition of claim 3, wherein the fragment of the ZAG protein consists of any one amino acid sequence selected from SEQ ID NOS: 1 to 18.

5. The pharmaceutical composition of claim 3, wherein the fragment of the ZAG protein consists of any one amino acid sequence selected from SEQ ID NOS: 19 to 22.

6. A cosmetic composition for improving scars, comprising a ZAG protein-derived peptide.

7. The cosmetic composition of claim 6, wherein the scars include hypertrophic scars, keloids, atrophic scars, or a combination thereof.

8. The cosmetic composition of claim 6, wherein the ZAG protein-derived peptide is a peptide consisting of an amino acid sequence of 10-mer or more and 30-mer or less, which includes a fragment of the ZAG protein.

9. The cosmetic composition of claim 8, wherein the fragment of the ZAG protein consists of any one amino acid sequence selected from SEQ ID NOS: 1 to 18.

10. The cosmetic composition of claim 8, wherein the fragment of the ZAG protein consists of any one amino acid sequence selected from SEQ ID NOS: 19 to 22


SEQUENCE LISTING (23 sequences)

SEQ ID NO: 1 - protein, 30 aa, Artificial Sequence
  ZAG protein-derived peptide
QENQDGRYSLTYIYTGLSKHVEDVPAFQAL

SEQ ID NO: 2 - protein, 30 aa, Artificial Sequence
  ZAG protein-derived peptide
GSLNDLQFFRYNSKDRKSQPMGLWRQVEGM

SEQ ID NO: 3 - protein, 30 aa, Artificial Sequence
  ZAG protein-derived peptide
EDWKQDSQLQKAREDIFMETLKDIVEYYND

SEQ ID NO: 4 - protein, 30 aa, Artificial Sequence
  ZAG protein-derived peptide
SNGSHVLQGRFGCEIENNRSSGAFWKYYYD

SEQ ID NO: 5 - protein, 30 aa, Artificial Sequence
  ZAG protein-derived peptide
GKDYIEFNKEIPAWVPFDPAAQITKQKWEA

SEQ ID NO: 6 - protein, 30 aa, Artificial Sequence
  ZAG protein-derived peptide
EPVYVQRAKAYLEEECPATLRKYLKYSKNI

SEQ ID NO: 7 - protein, 30 aa, Artificial Sequence
  ZAG protein-derived peptide
LDRQDPPSVVVTSHQAPGEKKKLKCLAYDF

SEQ ID NO: 8 - protein, 30 aa, Artificial Sequence
  ZAG protein-derived peptide
YPGKIDVHWTRAGEVQEPELRGDVLHNGNG

SEQ ID NO: 9 - protein, 30 aa, Artificial Sequence
  ZAG protein-derived peptide
TYQSWVVVAVPPQDTAPYSCHVQHSSLAQP

SEQ ID NO: 10 - protein, 30 aa, Artificial Sequence
  ZAG protein-derived peptide
GLSKHVEDVPAFQALGSLNDLQFFRYNSKD

SEQ ID NO: 11 - protein, 30 aa, Artificial Sequence
  ZAG protein-derived peptide
RKSQPMGLWRQVEGMEDWKQDSQLQKARED

SEQ ID NO: 12 - protein, 30 aa, Artificial Sequence
  ZAG protein-derived peptide
PFDPAAQITKQKWEAEPVYVQRAKAYLEEE

SEQ ID NO: 13 - protein, 30 aa, Artificial Sequence
  ZAG protein-derived peptide
CPATLRKYLKYSKNILDRQDPPSVVVTSHQ

SEQ ID NO: 14 - protein, 30 aa, Artificial Sequence
  ZAG protein-derived peptide
IFMETLKDIVEYYNDSNGSHVLQGRFGCEI

SEQ ID NO: 15 - protein, 30 aa, Artificial Sequence
  ZAG protein-derived peptide
ENNRSSGAFWKYYYDGKDYIEFNKEIPAWV

SEQ ID NO: 16 - protein, 30 aa, Artificial Sequence
  ZAG protein-derived peptide
APGEKKKLKCLAYDFYPGKIDVHWTRAGEV

SEQ ID NO: 17 - protein, 30 aa, Artificial Sequence
  ZAG protein-derived peptide
QEPELRGDVLHNGNGTYQSWVVVAVPPQDT

SEQ ID NO: 18 - protein, 30 aa, Artificial Sequence
  ZAG protein-derived peptide
AVPPQDTAPYSCHVQHSSLAQPLVVPWEAS

SEQ ID NO: 19 - protein, 15 aa, Artificial Sequence
  ZAG protein-derived peptide
APGEKKKLKCLAYDF

SEQ ID NO: 20 - protein, 15 aa, Artificial Sequence
  ZAG protein-derived peptide
KKLKCLAYDFYPGKI

SEQ ID NO: 21 - protein, 15 aa, Artificial Sequence
  ZAG protein-derived peptide
LAYDFYPGKIDVHWT

SEQ ID NO: 22 - protein, 15 aa, Artificial Sequence
  ZAG protein-derived peptide
YPGKIDVHWTRAGEV

SEQ ID NO: 23 - protein, 298 aa, Homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS